(19) United States
(12) Patent Application Publication
(10) Pub. No.: US 2020/0030100 A1
(43) Pub. Date: Jan. 30, 2020

(54) PATIENT SPECIFIC CARTILAGE DEFECT REPLACEMENT

(71) Applicant: Orthopedix, Inc., Louisville, KY (US)

(72) Inventors: Adam E. M. Eltorai, Louisville, KY (US); Ashok Seetharam, Louisville, KY (US); Vishal J. Thomas, Louisville, KY (US)

(21) Appl. No.: 16/045,713

(22) Filed: Jul. 25, 2018

(51) Int. Cl. A61F 2/30; A61F 2/08

(52) U.S. Cl. CPC A61F 2/30756; A61F 2/30942; A61B 2034/105; A61F 2002/30952; A61F 2/08

(57) ABSTRACT

Defects and disease in human cartilage may arise from osteoarthritis, aging and joint injury, and are a major cause of joint pain and chronic instability. A soft tissue implant defined from anatomical scans of unhealthy tissue provides a shape and contour for replacing only unhealthy tissue while leaving healthy tissue intact. Patient specific scans identify the region occupied by soft tissue such as articular cartilage, and image an implant shaped to fit only the compromised regions. Surgical excision of the compromised region provides a shaped region for receiving the shaped implant, thereby leaving as much native healthy tissue as possible while completely replacing the volume of excised tissue. 3-dimensional printing based on scans of the afflicted region therefore provides a patient specific implant to engage the void remaining from excision of the compromised cartilage

### BACKGROUND

Prosthetic appliances are surgical implants that replace natural skeletal structures in a patient. Natural skeletal structures such as bones, tendons and ligaments can be compromised by age, disease and traumatic injury, as well as other causes. Surgical replacement with an orthopedic implant attempts to duplicate the original cartilage or skeletal member so that the patient may continue to enjoy mobility and dexterity once provided by healthy skeletal members. Modern developments in CAD/CAM (computer aided design/computer aided manufacturing) has facilitated fabrication of these complex shapes.

One facet of prosthetic replacement efforts includes articular cartilage. Articular cartilage is a soft white gristle that covers the ends of bones and helps joints to move smoothly. It lines the ends of human joint surfaces and is composed of cells called chondrocytes with a matrix or scaffolding made of collagen and proteins. In healthy joints, this unique and durable material allows bones to move against one another with minimal friction. Cartilage defects arising from osteoarthritis, aging and joint injury are a major cause of joint pain and chronic instability. Without blood vessels, nerves, and lymphatics, mature cartilage resists challenges to healing.

### SUMMARY

A soft tissue implant defined from anatomical scans of unhealthy tissue provides a shape and contour for selectively replacing a region of only unhealthy tissue while leaving healthy tissue intact. Patient specific scans identify the region occupied by soft tissue such as articular cartilage, and image an implant shaped to fit only the compromised regions. Surgical excision of the compromised tissue provides a shaped region for receiving the fabricated implant, thereby leaving as much native healthy tissue as possible while completely replacing the volume of excised tissue. Three dimensional (3D) printing based on scans of the afflicted region therefore provides a patient specific implant to engage the void remaining from excision of the compromised cartilage.

A common treatment for advanced cartilage degeneration include joint and cartilage replacement surgery, but this procedure is highly invasive, complicated, and expensive. Although cell transplantation-based tissue engineering treatment for human cartilage repair was introduced almost two decades ago, current cartilage tissue engineering strategies cannot as yet fabricate new tissue that is indistinguishable from native cartilage with respect to zonal organization, extracellular matrix (ECM) composition, and mechanical properties. Furthermore, almost all current strategies of cartilage repair involve removal of healthy cartilage tissue around the lesion site to create artificial defects for further treatment or implantation. This procedure in fact causes additional necrosis to the existing cartilage tissue and it is believed to lead to ultimate cartilage degeneration and failure of implanted tissue.

Configurations herein are based, in part, on the observation that orthopedic implants are employed to replace human skeletal members which often interface with other prosthetics or natural bone, as in a moving or pivoting juncture. Rigid skeletal structures often form an articulation between bones held in a moveable arrangement with connective tissue. Resilient tissue such as cartilage often occupies a void between the moving bones, and acts as a buffer or cushion to mitigate impact and frictional forces which would otherwise be passed to the bones. This cartilage can be prone to disease and compromise, leading to encumbered movement of the articulated joint. Unfortunately, conventional approaches to cartilage repair often evacuate the void or remove a substantial portion of the cartilage, thus extracting healthy tissue along with compromised regions. Accordingly, configurations herein substantially overcome the shortcomings of overreaching extraction by defining a compromised region from a 3D scan of the afflicted anatomical area. A flexible implant based on the shape and contour of the compromised region allows a prosthetic repair that replaces only unhealthy tissue, leaving healthy tissue intact as the prosthetic complements the shape of the healthy tissue to occupy the void. In this manner, as much natural tissue is left in the void to encourage natural tissue regrowth, particularly when the prosthetic defines a scaffold structure for facilitating natural tissue regrowth.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a plan view of the soft tissue implant according to the approach herein;

FIG. 2 is a view of the prosthetic implant of FIG. 1 showing contour and attachment points;

FIG. 3 is a side view of the implant of FIG. 2;

FIG. 4 is a bottom view of the implant of FIGS. 2 and 3; and

FIG. 5 is a diagram of the fabrication process for generating an implant such as that in FIGS. 1-4.

### DETAILED DESCRIPTION

Depicted below is an example fabrication and implant procedure of the approach herein. The disclosed example depicts a typical skeletal structure for receiving the fabricated implant, however any suitable skeletal structure may be employed.

Configurations below employ 3D bioprinting, which is based on thermal inkjet rendering, and has substantial potential for technological advancement in the field of tissue engineering and regenerative medicine. With digital control, cells, scaffolds and growth factors can be precisely deposited to the desired two-dimensional (2D) and three-dimensional (3D) locations rapidly. Therefore, this technology is an ideal approach to fabricate tissues mimicking their native anatomic structures. The major advantage of 3D printing is that it could address some of the complexities of the tissues. For example, an implant could replicate some of the layered structure of the cartilage, or even the layered structure of the osteochondral tissue—in other words, the cartilage plus the underlying bone. Another advantage of 3D printing is that it may require less tissue handling than other approaches, and the automated rendering process facilitates sizing of implants.

Configurations herein present a system, method and apparatus for printing articular cartilage tissues for any suitable bone joint from MRI scan images of the patient. The proposed method involves replacing only the lost cartilage tissue area thereby eliminating any need to replace the entire joint, which is typically the goal in most existing treatment options.

Cartilage is classified in three types, elastic cartilage, hyaline cartilage and fibrocartilage, which differ in relative amounts of collagen and proteoglycan. Articular cartilage is hyaline cartilage on the articular surfaces of bones. As such, it lies inside the joint cavity of synovial joints, bathed in synovial fluid produced by the synovial membrane that lines the walls of the cavity.

One particular area of cartilage based joint disease includes the knee. Knee osteoarthritis is a common affliction that benefits from configurations herein. Untreated, it is usually a progressive degenerative disease in which the joint cartilage gradually wears away. The knee is also the most common area for cartilage restoration. Other areas benefiting from replacement intervention include the hip, ankle and shoulder. The cartilage here helps absorb forces throughout the knee and allows the knee joint to move smoothly. The cartilage surface can be damaged by trauma such as a sports injury. Normal use, including running, will not usually wear out the cartilage unless it has been previously injured or if the meniscus cartilage has been removed. Bone malalignment or obesity can also contribute to damage. The diseases of osteoarthritis and inflammatory arthritis can directly damage the cartilage surfaces as well. The most frequent symptom is a dull pain around or under the knee cap that worsens when walking down stairs or hills. A patient may also feel pain when climbing stairs or when the knee bears weight as it straightens. The disorder is common in runners and is also seen in skiers, cyclists, and soccer players. It occurs most often in young adults and can be caused by injury, overuse, parts out of alignment, or muscle weakness. Instead of gliding smoothly across the lower end of the thigh bone, the knee cap rubs against it, thereby roughening the cartilage underneath the knee cap.

When areas of cartilage are worn away or torn away, exposing underlying (subchondral) bone, conventional treatment may be designed to fill in the missing area or defect with healthy articular cartilage and provide new protection for the joint surface. A difficult challenge for orthopedic surgeons is the treatment of injured joint surface cartilage in the young and active patient. Localized damage to one of the surfaces of the knee joint can lead to degradation and destruction of the opposing healthy joint surface. While total knee replacement is often an excellent option in the older patient, in young active patients who are not candidates for knee replacement, there is a need to promote healing of these injured surfaces with healthy hyaline cartilage to avoid long term problems with degenerative arthritis. Configurations herein allow selective replacement to leave as much healthy tissue as possible to promote healing, and the implant may be constructed of synthetic cartilage, tissue scaffolding responsive to new tissue growth, and biocompatible metal to further promote healing.

FIG. 1 is a plan view of the soft tissue implant according to the approach herein. Referring to FIG. 1, the implant 150 occupies a region of soft tissue attached or adjacent to a healthy skeletal member 100. Based on a region of removed, diseased tissue, the implantation includes replacing the removed tissue with the rendered flexible implant 150 for occupying the same volume as the removed tissue. This allows removal of the compromised tissue while leaving the healthy tissue intact, facilitating healing and patient mobility by mitigating invasiveness.

In an example configuration, the disclosed procedure includes scanning the patient's diseased region using an MRI or other suitable scan, and generating the necessary DICOM files to enable proper segmentation of the injured area.

For segmentation, software suites such as VGStudio® or Materialise® Mimics may be used for the precise reconstruction of three-dimensional volume data sets using the images taken by the MRI scanner to visualization (in 3D and 2D).

A successful segmentation procedure yields a 3D model of the cartilage defect region as a .stl file, which may be further processed as necessary. Design optimization software such as Materialise 3-Matic or Blender are then used to accurately map out the shape and dimensions of the cartilage defect. The resulting model is employed in the successive steps discussed below.

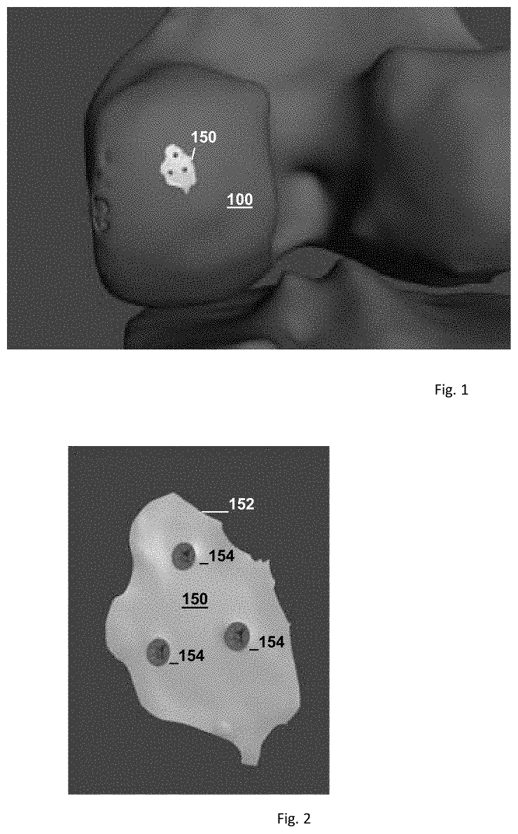

FIG. 2 is a view of the prosthetic implant of FIG. 1 showing contour and attachment points. Depending on the exact area and volume of diseased tissue (typically cartilage), the implant 150 defines a circumferential contour 152. Attachment members, such as screw 154, rods or anchors may be employed for attachment to a more rigid bone surface. Alternatively, adhesives or tethers such as sutures may be employed.

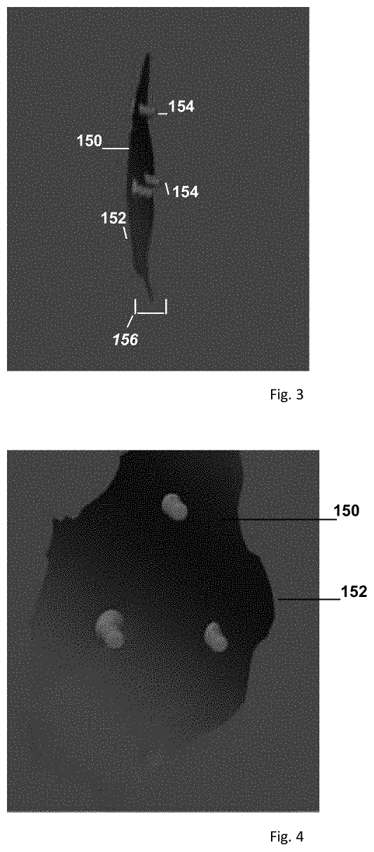

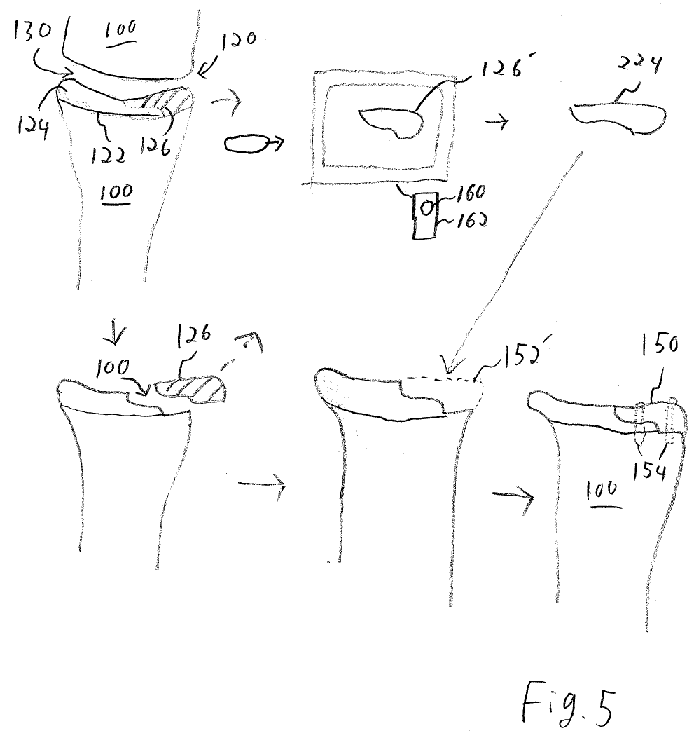

FIG. 3 is a side view of the implant of FIG. 2 illustrating a depth 156, in addition to the contour 152 and anchors 154. FIG. 4 is a bottom view of the implant of FIGS. 2 and 3.

FIG. 5 is a diagram of the fabrication process for generating an implant such as that in FIGS. 1-4. In FIG. 5, the method of soft tissue implant fabrication as disclosed herein includes receiving scan data 110 of an anatomical region 120 of resilient tissue 122 having portions of both healthy 124 and compromised 126 areas. Resilient tissue 122 such as cartilage often appears at an end of a more rigid skeletal structure 100, and at least partially occupies a void 130 defining an articulation region between moving bones. The resilient nature of the tissue 122 helps mitigate forces and friction of the moving bones 100.

The scan data 110 defines a shape of the compromised tissue, in which the compromised tissue represents only a portion of resilient tissue occupying the void 130 between the bones; healthy tissue occupies the remainder. Scan data may be from any suitable biomaterial scanning process, such as MRI (Magnetic Resonance Imaging) CT (Computer Axial Tomography, or CAT) The scan data 110 depicts a 3-dimensional region of articulated skeletal members represented as regions of rigid bone, structural tissue and soft tissue, such that the soft tissue occupies the void 130 between the articulated skeletal members

From the scan data, an analysis application 160 on a fabrication server 162 analyzes the scan data 110 and identifies, based on the received scan, portions of the compromised resilient tissue in the anatomical region 120. The analysis application 160 generates a 3D (3 Dimensional) image defining the compromised tissue 126′ as a distinct portion separate from the entire region 122 of resilient tissue that occupies the end of the bone 100 and/or void 130. The result is a 3D image that defines a complement to the healthy tissue in the void 130 following removal of the compromised resilient tissue portion. In practice, following a capture of the scan data 110, an excision procedure removes the compromised tissue portion 126, leaving a tissue void 140. Generally, the compromised resilient tissue 126 includes cartilage disposed in the void 130 between articulated skeletal members 100. A rendering application renders a flexible implant 224 sized similarly to the portions of compromised tissue 126. The flexible implant 224 is defined by contours of the compromised resilient tissue 126. This generates a flexible implant adapted for placement around undisturbed healthy tissue.

In particular configurations, the approach takes the form of an image processing and rendering computer system and attached 3-dimensional printing device defining an apparatus for fabricating the soft tissue implants. The system includes an interface configured to receive scan data of an anatomical region of resilient tissue having healthy and compromised areas, and a processing device having image processing logic for identifying, based on the received scan, portions of the compromised resilient tissue in the anatomical region. The processing device also includes rendering logic configured to generate a 3D (3 Dimensional) image defining the compromised tissue; and an extrusion nozzle responsive to the rendering logic to extrude a flexible implant corresponding to the compromised tissue, such that the flexible implant is adapted for placement around undisturbed healthy tissue.

In one configuration, the biocompatible rendering medium has the following characteristics:

Friction coefficient: 0.0025

Functioning contact pressure range—2 to 11 MPa

Thickness—2-4 mm

Equilibrium tensile modulus—1 to 30 MPa

Compressive Aggregate Modulus—0.4 to 1.5 MPa

Candidate materials may be synthetic (plastics, polymers, metals) or more biologic (scaffolding, transplanted cells, autologous, allograft, cultured cells). Specific materials having a desired blend of characteristics include the following:
Poly(ethylene glycol) dimethacrylate (PEGDMA), which after being bioprinted, has a compressive modulus of 395.73±80.40 kPa, which is close to the range of the properties of native human articular cartilage is a potential scaffolding material that can be seeded with human chondrocytes and used for direct cartilage repair.
PEG (Polyethylene glycol) (compressive modulus after bioprinting exceeds 0.5 MPa) hydrogel is another suitable scaffolding material. Cells to be used here are bone marrow or adipose tissue derived human mesenchymal stem cells (hMSC's). This is a one-step inkjet bioprinting approach involving PEG scaffold crosslinking, acrylated Arg-Gly-Asp (RGD) and acrylated matrix metalloproteinase (MMP) sensitive peptides covalent conjugation, hMSCs encapsulation, and layer-by-layer 3D tissue construction.

These biocompatible materials are selected in view of the following constraints imposed by specific activities:

Light jogging—7.7 MPa
Standing up—9.2 MPa
Stair climbing—10 MPa
One leg stance—6.7 MPa

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of soft tissue implant fabrication, comprising:
receiving scan data of an anatomical region of resilient tissue having healthy and compromised areas;
identifying, based on the received scan, portions of the compromised resilient tissue in the anatomical region;
generating a 3D (3 Dimensional) image defining the compromised tissue; and
rendering a flexible implant corresponding to the compromised tissue, the flexible implant adapted for placement around undisturbed healthy tissue.

2. The method of claim 1 wherein the compromised resilient tissue includes cartilage disposed in a void between articulated skeletal members.

3. The method of claim 1 further comprising:
removing the compromised tissue while leaving the healthy tissue intact; and
replacing the removed tissue with the rendered flexible implant for occupying the area of the removed tissue.

4. The method of claim 1 wherein the flexible implant is defined by contours of the compromised resilient tissue.

5. The method of claim 1 wherein the scan data depicts a 3-dimensional (3D) region of articulated skeletal members represented as regions of rigid bone, structural tissue and soft tissue, the soft tissue occupying a void between the articulated skeletal members.

6. The method of claim 2 wherein the scan data defines a shape of the compromised tissue, the compromised tissue representing a portion of resilient tissue occupying the void; and
generating the 3D image as a complement to the healthy tissue in the void following removal of the compromised resilient tissue portion.

7. The method of claim 1 wherein rendering the flexible implant further comprises 3D printing of a medium including at least one of synthetic cartilage, tissue scaffolding responsive to new tissue growth, and biocompatible metal.

8. An apparatus for fabricating soft tissue implants, comprising:
an interface configured to receiving scan data of an anatomical region of resilient tissue having healthy and compromised areas;
image processing logic for identifying, based on the received scan, portions of the compromised resilient tissue in the anatomical region;
rendering logic configured to generate a 3D (3 Dimensional) image defining the compromised tissue; and
an extrusion nozzle responsive to the rendering logic to extrude a flexible implant corresponding to the compromised tissue, the flexible implant adapted for placement around undisturbed healthy tissue.

9. The apparatus of claim 8 further comprising an extrusion printing device adapted to extrude a biocompatible medium responsive to a motor driven carriage and based on the rendering logic.
